(19) United States
(12) Patent Application Publication
(10) Pub. No.: US 2012/0310054 A1
(43) Pub. Date: Dec. 6, 2012

(54) BOLUS

(75) Inventor: Uzi Birk, Huddinge (SE)

(73) Assignee: DELAVAL HOLDING AB, Tumba (SE)

(21) Appl. No.: 13/520,187

(22) PCT Filed: Jan. 18, 2011
(86) PCT No.: PCT/EP2011/050635

§ 371 Date: Jul. 2, 2012

Related U.S. Application Data
(60) Provisional application No. 61/299,480, filed on Jan. 29, 2010.
(60) Provisional application No. 61/333,408, filed on May 11, 2010.

(30) Foreign Application Priority Data
Jan. 21, 2010 (SE) 1050068-4

(51) Int. Cl. A61B 5/07

(52) U.S. Cl. 600/302

(57) ABSTRACT

A bolus, for introduction into a ruminant animal's reticulum to register pressure signals transmitted through fluids therein, has a case unit adapted for immersion in the fluids. A mechanical amplifier element extends out from the case unit and is adapted to be surrounded by the fluids in the reticulum and absorb mechanical energy from the pressure signals. A guide part conveys mechanical energy from the mechanical amplifier element to a sensor module in the interior of the case unit. The sensor module transduces the pressure signals into electric signals from which a processing module extracts data representing body movements, a heart beat rate, a respiratory rate, a respiratory depth and/or stomach activity of the animal. A communication module in the case unit receives the data and transmits output radio signals reflecting the data.

### THE BACKGROUND OF THE INVENTION AND PRIOR ART

The present invention relates generally to monitoring of the physiological status of ruminant animals, such as dairy animals. More particularly the invention relates to a bolus according to the preamble of claim 1.

To accomplish an efficient and animal friendly livestock handling it is important that the animals' physiological state and health condition be monitored. Of course, to this aim, regular farmer's inspections and veterinary examinations can never be excluded. However, as a complement thereto and to provide an ongoing supervision, various automatic systems can be employed. One example of such a solution involves introducing a so-called bolus into the animal's stomach. More precisely, the bolus should preferably be placed in the reticulum, and normally the bolus has a weight of around 50-200 grams in order to not be forwarded through the digestive system. Once installed in the animal, the bolus may register internal body signals of the animal and report corresponding data to an external station via a radio interface.

U.S. Pat. No. 6,059,733 discloses a method of determining a physiological state of a ruminant animal using an ingestible bolus. The bolus includes a temperature sensor and a transmitter. Thus, various core body temperatures within the animal's stomach can be registered and reported to a remote receiver for mathematical analysis. As a result, the physiological state of the animal can be determined.

US 2007/0088194 describes a bolus for introducing into a ruminant animal's reticulum. The bolus contains an acoustic sensor for receiving acoustic signals emanated by various signal sources in the animal, such as the heart and respiratory organs, and output values indicative of the animal's health condition.

However, the body temperature alone as determined in the former reference does not provide an adequate basis for determining the animal's health condition. Moreover, to protect the acoustic sensor of the latter reference from the harsh environment represented by the stomach, the sensor is enclosed in an acoustic chamber of the bolus. Unfortunately, this also means that the sensor's sensitivity becomes comparatively low.

### SUMMARY OF THE INVENTION

The object of the present invention is therefore to alleviate these problems, and thus offer high-sensitivity registration of pressure signals in a ruminant animal's reticulum.

According to the invention, this object is achieved by the initially described bolus further containing a sensor module, a mechanical amplifier element and a guide means. The sensor module is included in the interior of the bolus' case unit. The sensor modole is configured to transduce pressure signals into electric signals, which form a basis for data representing body movements, a heart beat rate, a respiratory rate, a respiratory depth and/or stomach activity of the animal. The mechanical amplifier element extends out from the case unit. The mechanical amplifier elerent is adapted to be surrounded by the fluids in the reticulum and absorb mechanical energy from the pressure signals. The guide means is configured to convey mechanical energy from the mechanical amplifier element to the sensor module for processing.

The proposed bolus is advantageous because it combines high acoustic sensitivity with mechanical robustness and ample protection for the sensor.

According to one preferred embodiment of the invention the mechanical amplifier element has a substantially flat surface configured to be repositioned in response to the pressure signals. Further preferably, the substantially flat surface, in turn, is operatively connected to the guide means. Thus, also relatively small pressure variations can be registered accurately.

According to another preferred embodiment of the invention the substantially flat surface has an overall circular shape. Namely, this renders the design well suited for being tossed around in the reticulum without injuring the animal or interfering significantly with the digestive function.

According to further preferred embodiments of the invention the sensor module includes a piezoelectric sensor, a capacitive sensor, an inductive sensor or a microelectromechanical system (MEMS) accelerometer. A piezoelectric sensor is beneficial because it has relatively high strain sensitivity, is very rugged, has an extremely high natural frequency and has an excellent linearity over a wide amplitude range. Capacitive and inductive sensors are beneficial because they are simple and cost efficient. A MEMS accelerometer is advantageous, since such a sensor can be made very small-sized and may register movements in three dimensions. Additionally, the sensor can determine static pressures as well as extremely slow pressure variations.

According to additional preferred embodiments of the invention the sensor module includes an optical transmitter-receiver pair interconnected via an optical transmission path, where the transmission properties are variable in response to any displacements of the guide means. Such a variation may be accomplished through a pivotable mirror element connected to the guide means and having a reflective surface an angle of which is variable in at least one dimension relative to at least one of an incoming light path from the optical transmitter and an outgoing light path to the optical receiver. Hence, depending on the angle of the reflective surface different amounts of light from the transmitter reaches the receiver. The optical transmission path may further include optical fibers, for instance one or more optical fibers configured to supply light energy from the optical transmitter along said incoming light path, and one or more receiving fibers configured to receive light energy via said outgoing light path and transport the received light energy to the optical receiver. The employment of optical fibers renders the design flexible in terms of where the transmitter and receiver can be located. On the other hand, transmitting the light without dedicated conduits renders the design less complex and more cost efficient.

According to yet another preferred embodiment of the invention the communication module includes a radio interface configured to generate radio signals to represent the output signals. Hence, these signals may be externally received in a convenient and straightforward manner, and then be further processed and/or presented to a user.

According to still another preferred embodiment of the invention the case unit has a general cylinder shape with first and second short sides. The mechanical amplifier element is here arranged to extend from the first short side. Further preferably, the case unit likewise includes a ballast having such weight and position relative to the contents of the case unit that a center of gravity of the case unit is located closer to the second short side than the first short side. Thereby, the bolus is balanced by the ballast and the buoyancy such that the first short side with the mechanical amplifier element points in a general upward direction when the bolus is installed in the animal, and this in turn, improves the sensor's sensitivity.

According to another preferred embodiment of the invention the interior of the case unit contains a battery module configured to deliver electric power to the sensor module, the processing module and/or the communication module. Preferably, the battery module here constitutes at least a part of the ballast.

Further advantages, advantageous features and applications of the present invention will be apparent from the following description and the dependent claims.

### BRIEF DESCRIPTION OF THE DRAWINGS

The invention is now to be explained more closely by means of referred embodiments, which are disclosed as examples, and with reference to the attached drawings.

FIG. 1 schematically shows a bolus according to one embodiment of the invention;

FIG. 2 illustrates a ruminant animal carrying a proposed bolus in its reticulum; and

FIGS. 3a-e show embodiments of a proposed sensor module.

### DESCRIPTION OF PREFERRED EMBODIMENTS OF THE INVENTION

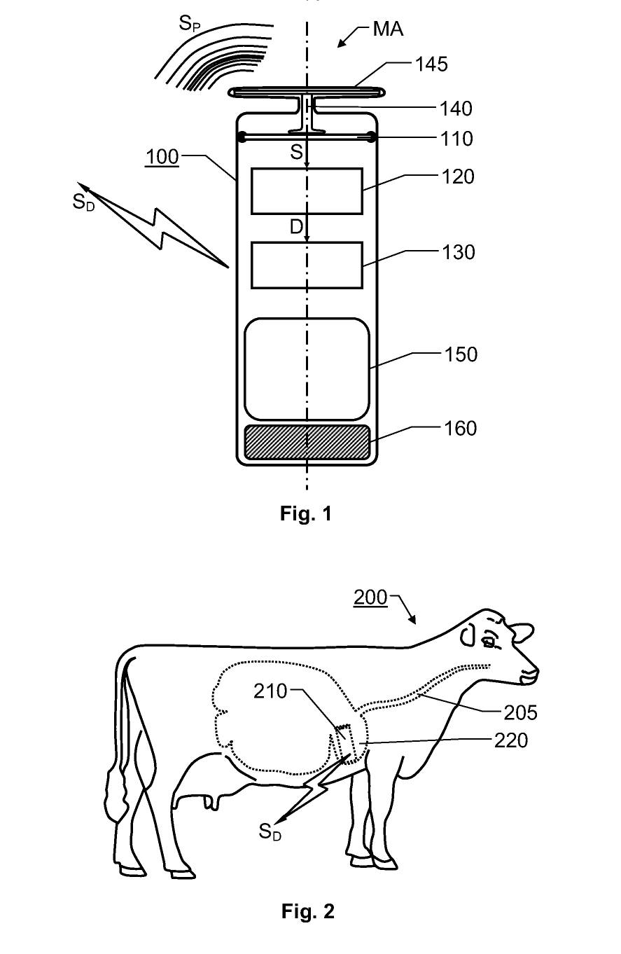

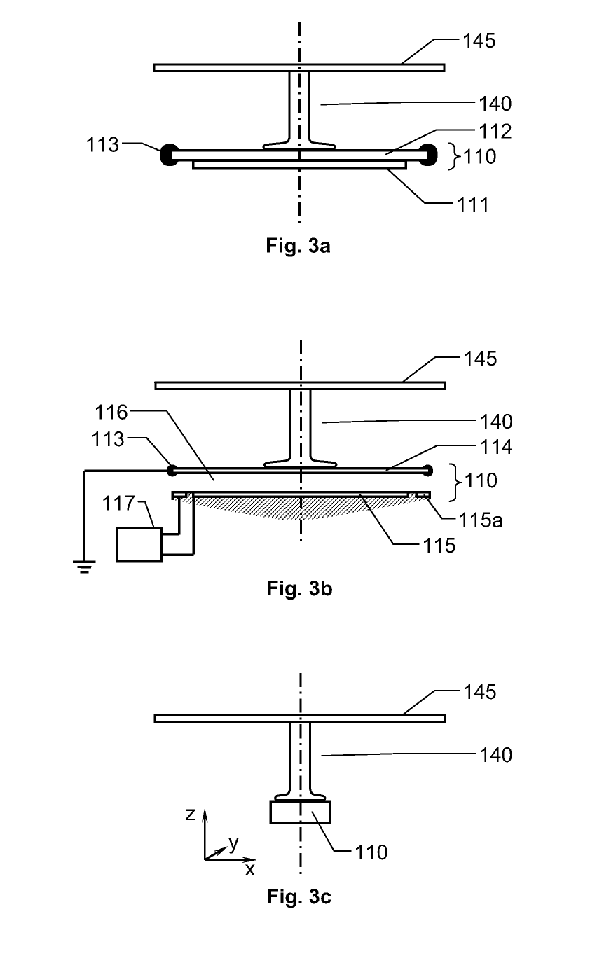

We refer initially to FIGS. 1 and 2, where FIG. 1 schematically shows a bolus according to one embodiment of the invention, and FIG. 2 illustrates a ruminant animal 200 carrying the proposed bolus 210 in its reticulum 220.

The bolus 210 is configured to register pressure signals S_{P} transmitted through fluids in the reticulum 220. To this aim, the bolus 210 has a case unit 100, which is adapted to be immersed in said fluids while preventing the fluids from reaching an interior of the case unit 100. The case unit 100 is preferably made of a polymeric material being transparent to radio waves, such as natural rubber, nylon, PVC or polystyrene. The interior of the case unit 100 contains a sensor module 110, a processing module 120 and a communication module 130.

A mechanical amplifier element MA extends out from the case unit 100. The mechanical amplifier element MA is adapted to be surrounded by the fluids in the reticulum and absorb mechanical energy from the pressure signals S_{P}. Preferably, the mechanical amplifier element MA has a substantially flat main surface 145, which is configured to be repositioned in response to the pressure signals S_{P} (essentially move up and down) and thus function as an energy receiver. For practical reasons it is further advantageous if the substantially flat surface 145 of the amplifier element MA has an overall circular shape. Namely, thereby for a given maximum area of the surface, the risks are minimized that the amplifier element MA injures the animal or interferes significantly with the digestive function when the bolus 210 is being tossed around in the reticulum 220. The substantially flat surface 145 is operatively connected to a guide means 140 that is configured to convey mechanical energy from the mechanical amplifier element MA to the sensor module 110. Thus, the substantially flat surface 145 and the guide means 140 are preferably relatively rigid, while a top part of case unit 100 towards the substantially flat surface 145 and the guide means 140 is flexible, so that any movements of the substantially flat surface 145 will cause the guide means 140 to influence the sensor module 110.

As an alternative to the substantially flat surface 145, the mechanical amplifier element MA may include a dome-shaped surface configured to receive and convey energy from the pressure signals S_{P} to the sensor module 110.

The sensor module is configured to transduce any pressure signals forwarded via the guide means 140 into electric signals S. These signals, in turn, form a basis for said data D. More precisely, the processing module 120 is configured to receive the electric signals S and extract data D there from. Here, the data D represent: body movements, a heart beat rate, a respiratory rate, a respiratory depth and/or stomach activity of the animal 200.

The communication module 130 is configured to receive the data D and transmit output signals S_{D} (e.g. in the form of radio waves) from the animal 200, which output signals S_{D} reflect the data D.

Thus, to attain an efficient output of data for further processing and/or presentation to a user, it is advantageous if the communication module 130 has access to a radio interface, which is configured to generate radio signals representing the output signals S_{D}. However, technically, alternative technologies are also conceivable, such as transmitting the output signals S_{D} via magnetic resonance. It is further preferable if the bolus 210 is equipped with an on/off switch that is controllable from outside the animal 200, e.g. via said radio interface of the communication module 130, so that the bolus 210 can be selectively activated/deactivated in a straightforward manner.

Anyhow, the case unit 100 preferably has a general cylinder shape because this facilitates the introduction of the bolus 210 into the animal 200 via the esophagus 205. Said general cylinder has first and second short sides, and the mechanical amplifier element MA is arranged to extend from the first short side (top side in FIG. 1). The length of the bolus 210 is preferably 75-145 mm and the width is preferably 20-60 mm.

It has been found that the sensor in the sensor module 110 has the best sensitivity if the first short side (where the mechanical amplifier element MA is located) points in a general upward direction when the bolus 210 is installed in the animal 200. To increase the probability that the bolus 210 is oriented this way, it is advantageous if the case unit 100 includes at least one ballast 150 and/or 160 having such weight and position relative to the contents of the case unit 100 that a center of gravity of the case unit 100 is located closer to the second short side than the first short side. This renders the bolus balanced by the ballast and the buoyancy, such that the first short side with the mechanical amplifier element points in a general upward direction when the bolus is installed in the animal. Thereby, the sensor attains good sensitivity.

A battery module 150 is arranged in the interior of the case unit 100. The battery module 150 is configured to deliver electric power to the sensor module 110, the processing module 120 and/or the communication module 130. Clearly, it is beneficial if the battery module 150 has an expected life being at least as long as the expected remaining lifespan of the animal 200 after having introduced the bolus 210. This means that the battery module 150 should have a considerable capacity, and may thus constitute at least a part of the ballast. The overall weight of the bolus 210 is preferably around 200-300 grams, and the battery module 150 typically represents a substantial portion thereof.

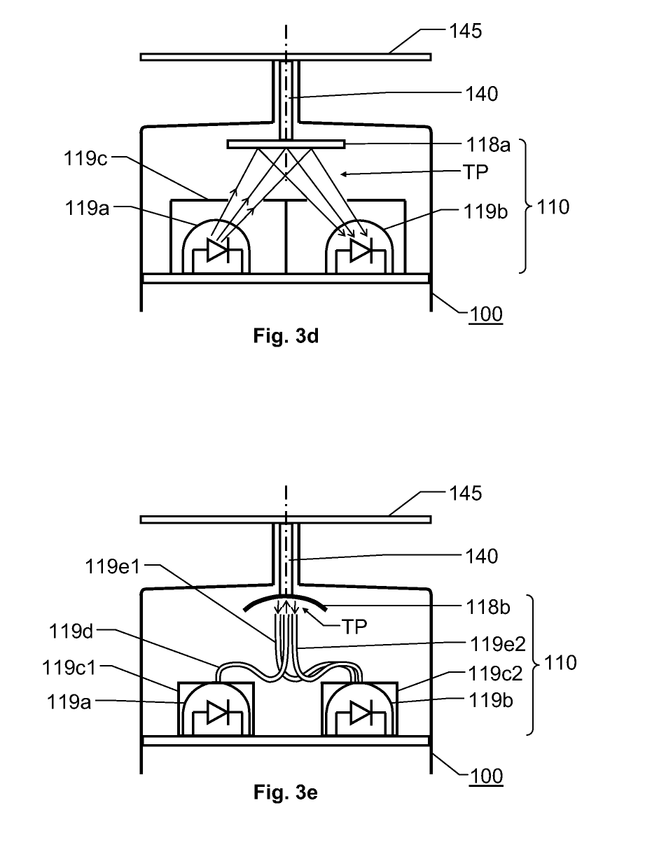

FIG. 3a shows a sensor module 110 according to a first embodiment of the invention, wherein the sensor module 110 includes a piezoelectric sensor 111, e.g. disc shaped. As can be seen in FIG. 3a, the guide means 140 is operatively connected to the sensor 111, such that any forces exerted on the mechanical amplifier element MA can be transported to the sensor 111. A support plate 112 (preferably of metal) is arranged on the opposite side of the sensor 111 to enable deformation of the sensor 111 in response to any forces applied via the guide means 140. To limit a maximum possible force exerted on the sensor 111 the sensor 111 is preferably connected to the case unit 100 via resilient members 113, e.g. of silicon. Alternatively, the sensor 111 may be held in the interior of the case unit 100 between a fixed support structure and a wave-shaped washer, which is configured to flex in response to a predefined force. Hence, any excessive external forces exerted on the sensor 111 can be absorbed by the wave-shaped washer, and consequently the sensor 111 is protected from mechanical overloading.

Due to the characteristics of the piezoelectric material in the sensor 111, a deformation thereof causes a voltage to be generated. Consequently, the variations in this voltage reflect pressure variations in the fluids of the reticulum 220. Since such pressure variations, in turn, may be the result of body movements, heart activities, activities of the respiratory organs and stomach activities, the voltage produced by the sensor 111 may form a basis for determining a heart beat rate, a respiratory rate, a respiratory depth and/or stomach activity of the animal 200.

FIG. 3b shows a sensor module 110 according to a second embodiment of the invention, wherein the sensor module 110 includes a capacitive sensor. Here, a first metal plate 114 is arranged over a second metal plate 115, and a dielectric material (e.g. air) separates the first metal plate 114 from the second metal plate 115. A detector circuit 117 is used for measuring a capacitance between the first and second metal plates 114 and 115. The detector circuit 117 may either be connected to the second metal plate 115 and a guard ring 115a arranged concentrically around the sensor metal plate 115 (as shown in FIG. 3b), or the detector circuit 117 may be connected to both the first and second metal plates 114 and 115. In any case, analogous to the above, movements of the first metal plate 114 causes variations in the capacitance, which in turn may reflect heart activities, activities of the respiratory organs and/or stomach activities.

More precisely, for a constant distance between the metal plates 114 and 115, any tilting from a horizontal orientation of the plate 114 relative to the plate 115 increases the capacitance. The guard ring 115a is beneficial because it reduces the amount sensor electrode boundary effects. Namely, the guard ring 115a renders the electric field between the metal plates 114 and 115 more homogeneous (inside the guard ring 115a).

The detector circuit 117 may be implemented according to various designs. For instance, if the first metal plate 114 is grounded and the detector circuit 117 is connected between the second metal plate 115 and the guard ring 115a, a relaxation oscillator with analog capacitance multiplier can be used to measure frequency variations, and thus determine the capacitance. If, on the other hand, the detector circuit 117 is connected between the first and second metal plates 114 and 115, an excitation source may charge the first metal plate 114 via a square-wave excitation signal. Simultaneously, charges manifested as a voltage on the second metal plate 115 are sampled to represent a capacitance measure. Here, the guard ring 115a may be used for differential capacitance measurements.

Alternatively, variations in inductive properties in response to displacements of one or more magnetic components can be studied. This means that the sensor module 110 instead includes an inductive sensor.

FIG. 3c shows a sensor module 110 according to a third embodiment of the invention, wherein the sensor module 110 includes a MEMS accelerometer configured to register acceleration and deceleration parameters. This type of component is advantageous because it can be designed as a so-called 3-axis sensor capable of registering deformations in three dimensions x, y and z (i.e. downwards/upwards as well as lateral movements in a plane). A 3-axis MEMS accelerometer detects acceleration and deceleration in three independent directions. Since gravitation constitutes one important example of acceleration (namely towards earth), the sensor can detect how it is oriented relative to earth with respect to each of said axes. Consequently, in addition to the above-mentioned pressure variations, the sensor may also determine static pressures. Additionally, extremely slow pressure variations can be registered accurately.

FIG. 3d shows a sensor module 110 according to a fourth embodiment of the invention. Here, the sensor module 110 includes an optical transmitter 119a, e.g. an IR LED, and an optical receiver 119b, e.g. an IR photodiode. The optical transmitter 119a and the optical receiver 119b are interconnected via an optical trans-mission path TP, where the transmission properties are variable in response to any displacements of the guide means 140.

The optical transmitter 119a is optically insulated from the optical receiver 119b by means of a shield 119c, such that light from the optical transmitter 119a may only reach the optical receiver 119b via a reflective surface of a mirror element 118a. The mirror element 118a, in turn, is connected to the guide means 140 and pivotable so that an angle thereof is variable in at least one dimension relative to at least one of an incoming light path from the optical transmitter 119a and an outgoing light path to the optical receiver 119b. Consequently, if the substantially flat surface 145 is oriented in a fully horizontal position, and therefore the guide means 140 is oriented vertically, the optical transmitter 119a transfers a maximal amount of light to the optical receiver 119b. Due to the mechanical relationship between the surface 145 and the guide means 140, any other positioning of the surface 145 will result in that less of the light energy per unit time emitted from the optical transmitter 119a is received by the optical receiver 119b.

FIG. 3e shows a sensor module 110 according to a fifth embodiment of the invention. Analogous to the design of FIG. 3d, the transmission properties of an optical transmission path TP between an optical transmitter 119a and an optical receiver 119b are here variable in response to displacements of the guide means 140 to which a mirror element 118b is connected. However, in the embodiment illustrated in FIG. 3e dedicated conduits in the form of optical fibers are used to convey light energy from the optical transmitter 119a to the optical receiver 119b. Specifically, a transmitting optical fiber 119d is configured to supply light energy from the optical transmitter 119a to a reflective surface of the mirror element 118b to constitute an incoming light path. Correspondingly, at least one receiving optical fiber, here exemplified by 119e1 and 119e2, is configured to receive light energy via an outgoing light path from the reflective surface of a mirror element 118b and transport the received light energy to the optical receiver 119b. Except for the optical transmission path TP embodied by the optical fibers 119d, 119e1 and 119e2, the optical transmitter 119a and the optical receiver 119b are optically insulates from one another by a respective shield 119c1 and 119c2.

Preferably, in the embodiments illustrated in FIGS. 3d and 3e, the optical transmitter 119a and the optical receiver 119b are controlled by a microprocessor, or similar control unit. The microprocessor is configured to control the optical transmitter 119a to emit light pulses. The optical receiver 119b is further associated with a detector circuitry, which may include a switched integrator and a comparator.

Under operation of the sensor nodule 110 the microprocessor causes the optical transmitter 119a to emit light pulses repeatedly. The microprocessor initiates a light pulse by activating the optical transmitter 119a. Simultaneously there with, the microprocessor starts time measurement. In response to the thus emitted light, the optical receiver 119b registers light energy and the switched integrator connected thereto produces a rising output. The comparator compares this output with a reference voltage, and when the output reaches the reference voltage, the comparator generates a signal to the microprocessor that stops the time measurement. The microprocessor then determines a pivoting angle of the guide means 140 based on a pulse width representing an interval between the start and stop of the time measurement. Here, a relatively wide pulse corresponds to a comparatively large pivoting angle, and a relatively narrow pulse corresponds to a comparatively small pivoting angle. The pulse width, in turn, constitutes a basis for the electric signal S.

The term “comprises/comprising” when used in this specification is taken to specify the presence of stated features, integers, steps or components. However, the term does not preclude the presence or addition of one or more additional features, integers, steps or components or groups thereof.

The reference to any prior art in this specification is not, and should not be taken as, an acknowledgement or any suggestion that the referenced prior art forms part of the common general knowledge in Australia, or any other country.

The invention not restricted to the described embodiments in the figures, but may be varied freely within the scope of the claims. For example, the guide means 140 is in the figures shown as being mechanically connected to the sensor module 110, but the mechanical energy from the mechanical amplifier element MA can of course instead be conveyed to the sensor module 110 through other means, including a magnetic coupling between the guide means 140 and the sensor module 110.

1-16. (canceled)

17. A bolus (210) for introducing into a ruminant animal's (200) reticulum (220) to register pressure signals (SP) transmitted through fluids therein, the bolus (210) comprising:
a case unit (100) adapted to be immersed in said fluids and prevent the fluids from reaching an interior of the case unit (100), the case unit having a top part with an exterior upper surface,
wherein, in use, the upper surface is in contact with the fluids;
a mechanical amplifier element (MA) extending out from the case unit (100), the mechanical amplifier element (MA) having an upper top surface (145) and an opposite lower bottom surface, the bottom surface being spaced apart from the upper surface of the case unit by a volume therebetween,
the mechanical amplifier element (MA) adapted to be surrounded by the fluids in the reticulum and absorb mechanical energy from the pressure signals (SP) within the fluids, the absorbed mechanical energy causing movement of the mechanical amplifier element (MA) in a direction towards the upper surface of the case unit (100),
wherein, in use, the fluids occupy the volume between the upper surface of the case unit and the bottom surface of the mechanical amplifier element (MA), contacting the upper and lower surfaces of the mechanical amplifier element (MA);
a sensor module (110) located within an interior of the case unit (100), the sensor module (110) being configured to transduce a pressure signal into an electric signal (S) forming a basis for data (D);
a guide part (140) extending through the upper surface of the case unit (100), the guide part operatively connected to the bottom surface of the mechanical amplifier element (MA) to convey the movement of the mechanical amplifier element (MA) as the pressure signal to the sensor module (110);
a processing module (120) located within the interior of the case unit (100) and operatively connected to the sensor module, the processing module (120) configured to extract the data (D) from the sensor module (110) resulting from the pressure signal from the mechanical amplifier element (MA), the data (D) representing at least one of i) body movements, ii) a heart beat rate, iii) a respiratory rate, iv) a respiratory depth, and v) stomach activity of the animal (200); and
a communication module (130) located within the interior of the case unit (100) and configured to receive the data (D) from the processing module and transmit output signals (SD) from the animal (200), which output signals (SD) reflect the data (D).

18. The bolus (210) according to claim 17, wherein,
the upper top surface of the mechanical amplifier element (MA) comprises a substantially flat surface (145) configured to be repositioned, moving in the direction towards and in a direction away from the upper surface of the case unit, in response to the pressure signals (SP), and
the repositioning of the substantially flat surface (145) is transmitted by the guide part (140) to the sensor module (110).

19. The bolus (210) according to claim 18, wherein the substantially flat surface (145) has an overall circular shape.

20. The bolus (210) according to any claim 17, wherein the sensor module (110) comprises a piezoelectric sensor (111).

21. The bolus (210) according to claim 17, wherein the sensor module (110) comprises a capacitive sensor (114, 115, 115a, 116).

22. The bolus (210) according to claim 17, wherein the sensor module (110) comprises an inductive sensor (114, 115, 116).

23. The bolus (210) according to claim 17, wherein the sensor module (110) comprises a microelectromechanical system accelerometer configured to register acceleration and deceleration parameters.

24. The bolus (210) according to claim 17, wherein the sensor module (110) comprises an optical transmitter (119a) and an optical receiver (119b), the optical transmitter (119a) and the optical receiver (119b) being interconnected via an optical transmission path (TP) having transmission properties that are variable in response to any displacements of the guide part (140).

25. The bolus (210) according to claim 24, wherein the optical transmission path (TP) comprises a pivotable mirror element (118a, 118b) connected to the guide part (140) and having a reflective surface with an angle variable in at least one dimension relative to at least one of i) an incoming light path from the optical transmitter (119a), and ii) an outgoing light path to the optical receiver (119b).

26. The bolus (210) according to claim 25, wherein the optical transmission path (TP) comprises:
at least one transmitting optical fiber (119d) configured to supply light energy from the optical transmitter (119a) along the incoming light path, and
at least one receiving optical fiber (119e1, 119e2) configured to receive light energy via the outgoing light path and transport the received light energy to the optical receiver (119b).

27. The bolus (210) according to claim 17, wherein the communication module (130) comprises a radio interface configured to generate radio signals to represent the output signals (SD).

28. The bolus (210) according to claim 17, wherein the case unit (100) further comprises a bottom part and a side part extending between the top and bottom parts, the top and bottom parts being short as compared to the side part.

29. The bolus (210) according to claim 28, wherein the case unit (100) further comprises a ballast (150, 160) having a weight and a position relative to other contents of the case unit (100) that a center of gravity of the case unit (100) is located closer to the bottom part than to the top part.

30. The bolus (210) according to claim 29, wherein the interior of the case unit (100) further comprises a battery module (150) configured to deliver electric power to at least one of i) the sensor module (110), ii) the processing module (120), and iii) the communication module (130).

31. The bolus (210) according to claim 30, wherein the battery module (150) constitutes a part of the ballast.

32. The bolus (210) according to claim 17, wherein the bolus is configured for a cow.

33. The bolus (210) according to claim 28, wherein,
the mechanical amplifier element (MA) has an overall circular shape,
the top and bottom surfaces of the mechanical amplifier element (MA) extend beyond the side part extending between the top and bottom parts of the case unit, and
the volume occupied by the fluids between the upper surface of the case unit and the bottom surface of the mechanical amplifier element (MA) has an overall circular shape extending from to the side part of the case unit.

34. The bolus (210) according to claim 28, wherein,
the guide part is centrally located within the overall circular shape of the mechanical amplifier element (MA).

35. The bolus (210) according to claim 34, wherein,
the top part of the case unit is flexible, and
the top surface of the mechanical amplifier element (MA) and the guide part are relatively rigid in comparison to the top part of the case unit.

36. The bolus (210) according to claim 28, wherein,
the mechanical amplifier element (MA) has an overall circular shape,
the top and bottom surfaces of the mechanical amplifier element (MA) toward the side part of the case unit,
the volume occupied by the fluids between the upper surface of the case unit and the bottom surface of the mechanical amplifier element (MA) has an overall circular shape extending towards the side part of the case unit,
the top part of the case unit is flexible, and
the top surface of the mechanical amplifier element (MA) and the guide part are relatively rigid in comparison to the top part of the case unit.
